(19) United States
(12) Patent Application Publication
(10) Pub. No.: US 2009/0291189 A1
(43) Pub. Date: Nov. 26, 2009

(54) Highly concentrated plant protein preparation and method for producing the same

(76) Inventors: Christian Zacherl, Freising (DE); Peter Eisner, Freising (DE); Claudia Pickardt, Freising (DE); Katrin Hasenkopf, Freising (DE)

Correspondence Address: BREINER & BREINER, L.L.C., P.O. BOX 320160, ALEXANDRIA, VA, 22320-0160, US

(21) Appl. No.: 12/308,692

(22) PCT Filed: Jun. 12, 2007
(86) PCT No.: PCT/DE2007/001025

§ 371 Date: Jan. 15, 2009

(30) Foreign Application Priority Data
Jun. 30, 2006 (DE) 10 2006 030 243.5

(51) Int. Cl. A23D 9/00; C07K 14/00; A23J 1/00; C07K 1/00

(52) U.S. Cl. 426/601; 530/350; 530/402; 426/656

(57) ABSTRACT

The invention relates to a protein preparation and a method for the production thereof. The protein preparation includes protein particles. A protein particle includes several plant protein molecules with at least one hydrophilic and at least one hydrophobic moiety each. The protein molecules are bound to each other in such a manner that the protein particle has a mainly hydrophobic surface. The preparation allows production of foodstuffs that are rich in protein, that have a pleasant taste, that do not appear to be dry, solid, hard or crumbly, that do not cause increased saliva production and that are easy to swallow.

### FIELD OF TECHNICAL APPLICATION

The invention relates to a preparation for the highly concentrated enrichment of foodstuffs with plant protein, a method for the production of the preparation and the description of the use of the preparation in foodstuffs.

### PRIOR ART

The use of plant proteins in foodstuffs is increasingly gaining importance. In addition to their good eco-efficiency and their low production costs, a range of plant proteins is distinguished by a health-promoting effect. For example, the cholesterol-lowering effect of soya protein or the blood sugar-regulating effect of lupin protein is already described in several publications. On the part of the FDA (Food and Drug Administration), soya protein has a health claim which states that the intake of at least 25 g soya protein per day significantly reduces the risk of contracting cardiovascular diseases. There are new studies according to which lupin protein also reduces the cholesterol level. To effectively lower the cholesterol level, sufficient amounts of biofunctional proteins must be present in the food, i.e. proteins influencing bodily functions and therefore promoting health. These must be added to the foodstuffs, in order to be able to take in the necessary dose with daily food. However, this is difficult without, in so doing, impairing the sensory quality of the foodstuffs.

Commercially available plant protein preparations have good emulsifying and foam-forming characteristics, a high water-absorptive capacity and a high gel strength. Owing to the high viscosity of the protein solutions, only a maximum of 5% soya protein can be worked into a drink which is still accepted from a sensory point of view. To achieve the bioactive effect of 25 g protein, at least 0.5 litres of the protein drink would have to be drunk, which is often refused by users.

The marked water binding of plant proteins brings it about that foodstuffs, into which plant proteins are worked in in larger quantities, appear to be very solid, dry and brittle. The proteins bind the water in the foodstuffs, lead to an unpleasant dry sensation in the mouth due to the high saliva production in the mouth, and are difficult to chew. It is therefore difficult to produce foodstuffs which are pleasant to the senses, with a creamy sensation in the mouth and with a high protein content.

In addition, there are groups in the population who have difficulties in taking in proteins sufficiently. People with difficulties in swallowing or difficulties in chewing and older people generally often do not take in nutrients sufficiently, which not infrequently leads to malnourishment. The undersupply with proteins which, as is known, frequently occurs in this group of people, often lies in a low provision of products rich in protein, which are easy to chew and/or to swallow and taste good. Protein preparations which are easy to swallow in high concentrations and can therefore be used for protein enrichment of foodstuffs for the groups of people mentioned are unknown up to now.

Furthermore, several plant protein preparations have an unpleasant aftertaste, because they contain secondary plant compounds such as for example phenolic compounds or bitter substances. The improvement to this circumstance is also to be taken into consideration when a preparation with good sensory characteristics is to be provided.

It is an object of the present invention to provide plant protein preparations and a method for the production, by which it is made possible to work large amounts of plant proteins into foodstuffs, without negatively influencing the sensory, techno-functional or haptic characteristics.

### DESCRIPTION OF THE INVENTION

The problem is solved by a protein preparation and the method of claims 1 and 22. Advantageous developments of the preparation and of the method can be seen from the sub-claims and from the following description.

The preparation according to the invention consists of protein particles, with a protein particle consisting of a plurality of plant protein molecules with at least one hydrophilic and at least one hydrophobic moiety each. The protein molecules are bound to each other in such a manner that the protein particle has a hydrophobic surface.

Advantageously, the protein molecules are bound to each other in such a manner than the protein particle has a rounded, in particular a globular or spherical shape.

Particularly advantageously, the protein molecules of the protein particle are bound to each other in the form of a sphere, such that the hydrophilic moieties of the protein molecules project into the interior of the sphere and the hydrophobic moieties of the protein molecules project into the exterior. In this form, the proteins are not water-soluble and do not have any marked techno-functional characteristics. In this form, they can not be used as emulsifiers or foaming agents for foodstuff applications.

The particles can be formed to be so stable that their spherical shape is also not destroyed by homogenisation at 10⁸ Pa (1000 bar). This stable arrangement can be achieved with proteins from various raw plant materials.

Advantageously, a protein particle has a protein content of >75% by weight, preferably >85% by weight of the dry mass. The particles in the preparation according to the invention are not soluble in water in the neutral range (pH value between 6.5 and 7.5) at 20° C., and have an average particle size of <50 μm, advantageously <10 μm.

Surprisingly, with the described preparation, from an aqueous suspension of the preparation with a dry substance content of 5% by weight, a dry substance content of over 30% by weight, particularly advantageously over 35% by weight can only be set by mechanical separation steps such as pressing or centrifuging. In contrast to this, water-soluble protein preparations according to the prior art can be concentrated by the said methods at a maximum to protein contents of 20% by weight.

Through the said structure with the hydrophobic layer lying externally, the plant protein scarcely binds water, for which reason saliva is scarcely required for the consumption and swallowing of the mechanically dewatered protein substance, even with protein contents of over 35% by weight. A soft, creamy sensation is produced in the mouth, so that the consumption of the mechanically dewatered, moist preparation is a pleasant sensation. Even people with difficulties in swallowing can consume foodstuffs enriched with this protein preparation.

A particularly soft sensation in the mouth occurs when the oil content in the preparation, in relation to the dry substance content, exceeds values of 1% by weight, particularly advantageously values of 5% by weight, but less than 20% by weight (method of determination according to Caviezel, AOAC® PEER_VERIFIED METHOD PVM 4:1997). In such oily preparations, the oil is deposited onto the particle surfaces with a thin layer, so that the particles can bind even less water. In addition, the thin oil layer has the advantage that it functions as a separating means between the protein particles and therefore increases their mobility. The sensation in the mouth is particularly soft and pleasantly creamy. The oil coating of the particles preferably consists of valuable plant oils with a content of unsaturated fatty acids >30% by weight in the oil. The use of protein and oil from the same plant genus is particularly efficient here for the reduction of the water binding of the preparation.

Preparations with the described oil content make it possible, in addition, to introduce lipophilic components into the preparation, such as for example beta-carotine, flavourings or biofunctional substances, which leads to a combined biofunctional foodstuff.

The preparation according to the invention is worked into foodstuffs preferably as a moist substance or suspension with a water content of 50 to 80% by weight, which makes a suspending of the particles in water during the production of foodstuffs superfluous and therefore saves costs.

Further novel preparations can be produced from the preparation according to the invention. A particularly soft and creamy preparation is obtained when the aqueous protein preparation is mixed with more than 20% by weight oil, particularly preferably with more than 40% by weight, and the water contained in the preparation is subsequently separated (e.g. in a drier, centrifuge). Hereby, the water contained in the moist preparation can be successfully replaced largely by oil. The preparation, then consisting predominantly of oil and protein, can be worked into foodstuffs which contain no or only little water, i.e. their residual water content lies below 10%, preferably below 5%. Examples of such foodstuffs are chocolate, chocolate fillings, various water-free savoury or sweet spreads, such as for example nut-nougat creams, and baked goods and confectionary products.

Leguminosae or oilseeds can be used as raw plant materials. The use of sunflower, soya, field bean, pea or lupin seeds is particularly advantageous. Particularly with the use of lupin, and here in particular of Lupinus angustifolius, highly effective biofunctional foodstuffs can be produced with the preparation, for example for lowering cholesterol. A preparation of sunflower seeds has the advantage that, according to the current state of knowledge, the protein does not trigger any allergic reactions in humans and can therefore also be consumed by people with a nut or lupin allergy.

With the preparations, protein-rich foodstuffs are to be able to be produced which taste good, do not appear to be dry, solid, hard or crumbly, do not cause increased saliva production and are easy to swallow. In particular, the plant protein preparations are to be made such that, above all, older people and people with difficulties in swallowing or problems with chewing are able to consume the protein-rich foodstuff without difficulty and are therefore able to take in sufficient amounts of protein. In addition, with such a preparation, biofunctional foodstuffs could be produced with a sufficiently high content of effective proteins to achieve the desired effect.

The protein preparations can not only be used for the highly concentrated enrichment of foodstuffs with proteins, but also in order to alter the rheological characteristics in foodstuffs, to reduce the effort when chewing and to reduce the sliding friction on the tongue and on the palate. Through the addition of this preparation, the foodstuffs are given a softer sensation in the mouth, which increases the enjoyment factor.

Some foodstuff applications are described below, which can be produced by the addition of the preparation according to the invention into the recipe. In addition, further applications are also conceivable, in which the preparation can be used.
spreads (sweet or savoury)
specialty salads
spreadable sausage goods (liver sausage, spreadable smoked pork sausage, spreadable minced meat sausage)
cream cheese and soft cheese
chocolate and chocolate fillings
desserts, creams, cakes
drinks
sauces
dietary foodstuffs
icecream

In aqueous foodstuffs, the preparation can be used as a suspension in water with a water content of 50 to 80% by weight. Hitherto, both preparations without oil (oil content below 3%) and also products with oil contents between 7 and 13% oil were used. Of course, preparations with other oil contents are also able to be produced.

In the proposed method for the production of the preparation from plant proteins, plant proteins which have at least one hydrophilic and at least one hydrophobic moiety each are dissolved or suspended in a solvent with a first ionic strength and a first temperature, possibly with stirring, whereby a solution or suspension is obtained. Through a simultaneous lowering—taking place in short period of time (0.1 to 5 seconds)—of the first ion concentration and of the first temperature (environment change from warm to cold and from rich in salt to low in salt), a spontaneous change takes place in the tertiary structure (folding over/everting of the proteins) and a binding of the proteins to each other. The protein particles forming here from the individual (folded over/everted) proteins have a mainly hydrophobic surface. The protein particles precipitate out of the solution.

Compared with the described method, according to the prior art protein particles are obtained by thermic or chemical precipitation methods (e.g. by acid). Here, a partial gel formation or agglomerate formation of the proteins occurs via disulphide bridges, and the formation of water-insoluble particles is brought about. Certainly, the arrangement of the proteins in these particles is, however, random and very different particle sizes occur. In particular, no spherical particles with a hydrophobic surface are produced.

The solvent used for the production of the preparation is preferably aqueous, but can also have an alcoholic component.

In a preferred method, the first ionic strength and the first temperature of the solvent are reduced in that the solution or suspension, consisting of the solvent and the proteins, is mixed into a second solvent with a second temperature and a second ionic strength. Preferably, the protein solution/suspension is sprayed in through nozzles into the second solvent. Alternatively, a continuous precipitation can take place by mixing the solution or suspension with the second solvent in a mixing section.

In a particularly preferred embodiment of the method, the first temperature is between 25° C. and 40° C. and is lowered to below 10° C. The lowering is brought about for example by spraying the solution or suspension in through nozzles into the second solvent, which has a temperature of <4° C.

In a further preferred embodiment, the first ionic strength of the solvent corresponds to the ionic strength of a sodium chloride solution with a salt content of 1 to 4% by weight, preferably 2% by weight. This ionic strength is preferably reduced by a factor 2 to 50, particularly advantageously by a factor 3 to 10.

The protein solution or protein suspension is preferably mixed with the second solvent in the mixture ratio 1:3 to 1:12.

The simultaneous and spontaneous lowering, taking place in a short time, of the ionic strength and of the temperature in the solution or suspension constitutes an essential feature of the invention. In addition to the described method, further technical methods are also conceivable for the production of the preparation. A rapid lowering (taking place within 0.1-10 seconds) of the ion concentration can possibly also be achieved by other methods, for example by electrochemical methods, osmosis/ultrafiltration, dialysis or the addition of chemicals.

A rapid lowering of the temperature can also be achieved for example by the addition of ice or ice and water or by the introduction of liquid nitrogen.

Preferably, the proteins for the production of the protein preparation according to the invention are obtained by several method steps depending on the degree of processing of the raw materials. Proceeding from raw plant materials such as seeds or parts of seeds from oilseeds or leguminosae, and from a degree of pre-processing, the following steps can be necessary or are carried out:
Comminuting the raw plant materials for example into flakes or flour.
Pre-extraction in one or more steps of undesired water-soluble substances, e.g. sugar or bitter substances, by suspension of the comminuted or non-comminuted raw material in water in an environment of poor protein solubility. This can be set for example by a pH value <6 and/or by a low temperature <10° C. Undesired, water-soluble components are then separated by mechanical separation methods. The raw materials which are thus purified can be further processed.
Suspending the comminuted or non-comminuted, purified or non-purified raw materials in a, preferably aqueous, extraction agent and extracting of the proteins. Here, the proteins dissolve. In addition to purely aqueous extraction agents, extraction agents with an alcoholic component, for example between 10 and 80%, are also able to be used. Advantageous environmental conditions have a temperature >10° C., better >20° C., and a solid/liquid ratio (s:l) of 1:5 to 1:20.
Obtaining the extract containing protein by removing insoluble components such as fibres. Mechanical methods such as centrifuging, decanting, separating or filtering are suitable for this.

The obtained protein extract can be used directly for the production of the protein preparation. However, it is also possible to isolate the proteins from the extract, in order to then prepare with the isolated proteins a new solution or suspension for the production of the protein preparation. For example, a drying of the extract would be possible here. A concentrating or a diluting of the extract is also possible.

The size of the protein particles is dependent on the protein concentration in the extract or in the solution/suspension, the different between the first and second temperature and the difference of the ionic strengths of the solution/suspension. In particular, in a preferred method it can be set by the solution/suspension mixture ratio to precipitation medium (second solvent, e.g. cold, low-salt water). The protein content of the extract is influenced by washing off of sugars, separation of fibres, and possibly extraction of oil.

Following on from the precipitation of the protein particles, the protein particles are separated from the solvent surrounding them by a mechanical separation method. Advantageously, a re-washing of the protein particles takes place, and a separation of the washing water.

A protein preparation with a particularly pleasant and/or neutral taste is obtained, when the proteins provided for the production of the protein particles have as small a component of phenolic compounds as possible, because these have partially unpleasant or predominant taste characteristics. This can take place by a corresponding choice of the raw materials or else by the application of an additional method for phenol depletion.

The inventors have recognized that surprisingly the proportion of extracted phenolic compounds in a protein extract is reduced when, instead of water, an extraction agent containing salt is used. Furthermore, through the salt content in addition the oxidation of extracted phenols is suppressed. Non-oxidized phenols do not attach themselves onto the proteins and can be removed from the extract by mechanical separation methods for example. A further suppression of the oxidation of extracted phenols can be achieved in addition by the setting of a pH value <8 in the extraction agent.

Preparatory steps are then carried out depending on the extent of pre-processing of the raw materials which contain phenol, i.e. solution or suspension of the comminuted or non-comminuted raw materials and/or pre-extraction of undesired, water-soluble substances. The proteins are then extracted in an extraction agent, with an ionic strength being set in the extraction agent, which corresponds to a solution with a common salt content of over 0.5% by weight, particularly over 2% by weight. Through the salt content (characterized by the ionic strength), the co-extraction of phenolic compounds, i.e. the (undesired) dissolving of the phenolic compounds, is reduced compared with a salt-free or low-salt solution. With a common salt content of over 0.5% by weight, particularly over 2% by weight, a reduction of the component of the co-extracted phenolic compounds by more than 20% can be achieved. Alternatively, other salts can be used, with the ionic strength of the salt solution then being comparable with the above-mentioned common salt solution. A further advantage which results through the use of an extraction agent with a salt content as described above is a reduction, already able to be established, of the oxidation of the co-extracted phenolic compounds compared with a salt-free extraction agent. Furthermore, interactions of the phenols with the proteins, if necessary, can be further suppressed, if the temperature in the extraction agent is kept below 30° C., better still below 20° C., with, however, the protein extraction becoming poorer as the temperature decreases.

By means of mechanical methods such as centrifuging, separating, decanting or filtering, insoluble components such as fibres are removed from the extract. The extract additionally contains a proportion of non-oxidized, co-extracted phenolic compounds. These can be separated from the larger proteins by a mechanical separation method, in particular ultrafiltration, so that a low-phenol protein extract is obtained.

The low phenol protein extract which is obtained can be used directly for the production of the protein preparation. However, it is also possible to isolate the proteins from the extract, in order to then prepare a new solution or suspension with the isolated proteins for the production of the protein preparation. For example, a drying of the extract would be possible here. A concentrating or a diluting of the extract is also possible.

By setting a pH value <8.5 in the extraction agent the oxidation of nevertheless dissolved, phenolic compounds can be further suppressed. The non-oxidized phenolic compounds do not attach themselves onto the proteins and can be separated from the protein extract by mechanical separation methods. The obtained protein extract thereby has an even lower phenol content.

In the case of the extraction of proteins from sunflower seeds or fractions from sunflower seeds, in a particularly advantageous embodiment of the method, an increased salt concentration is set. For this, 3 to 20% by weight sodium chloride is added to the water which is used. In addition, the pH value is lowered, e.g. by the addition of an acid, to a value <7.0, better to a value <6.3. Thereby, the oxidation of secondary plant substances (in particular phenol carboxylic acids) can be suppressed and the attaching of these plant substances onto dissolved proteins can be prevented. When these substances are not oxidized, they are present freely dissolved in the extract and can be separated from the larger protein molecules mechanically, e.g. by ultrafiltration.

In a particularly advantageous application of the method, a highly concentrated sodium chloride solution >0.5 mol/l, preferably >1.5 mol/l, is used as the extraction solvent for the extraction of the protein from de-oiled oilseed flours. Surprisingly, in the protein extraction from sunflower flour with such a salt solution, it is found that here the co-extraction of undesired accompanying substances can be considerably reduced. For example, the content of co-extracted secondary plant substances which are disadvantageous from the sensory aspect (in particular phenol carboxylic acid derivatives), with the use of a sodium chloride concentration of 3 to 20% by weight, particularly advantageously of 10 to 14% by weight, are reduced by up to 90%.

Alternatively, with each method step, other salts can be added instead of common salt, with the ionic strength being comparable with the respective above-mentioned common salt solutions.

Instead of sunflower seeds, of course other raw materials containing phenol can also be used, for example rape, lupin.

In summary, the described method of phenol depletion in the extraction of proteins shows several steps by which the phenol content in the protein extract is lowered:
Reduction of the co-extraction of phenolic compounds by an increased salt content in the extraction agent;
reduction of the oxidation of nevertheless co-extracted phenolic compound, and hence avoidance of the irreversible attachment of the phenolic compound to the proteins, already by the increased salt content;
further reduction of the oxidation of nevertheless co-extracted phenolic compounds, and hence avoidance of the irreversible attachment of the phenolic compound to the proteins, by corresponding setting of a pH value <8.5, preferably however <7.0, in particular preferably <6.3;
separation of the non-oxidized phenolic compounds dissolved in the extraction agent, by physical methods, for example mechanical separation or adsorption.

A protein preparation which is produced from a low-phenol protein extract produced in this way has a very low concentration of bitter-tasting, phenolic compounds and therefore has a particularly neutral taste. It can therefore also be obtained from raw substances rich in phenol and nevertheless be worked in high concentrations into various foodstuffs.

### Example Embodiment

A simple example embodiment for the production of a product according to the invention is described below:

1) Stirring in of comminuted raw plant material containing protein (e.g. lupin flakes or lupin flour) in H₂O with a salt content of 2% by weight NaCl at a temperature of 40° C. Ratio of raw plant material to extraction agent 1:10.
2) Setting the pH value to 6.8; stirring at 100 RPM for 20 minutes at 40° C.
3) Centrifuging for 5 minutes at 20000 g
4) Discarding the precipitate (deposit, sediment)
5) Transferring the extract (40° C.) with a hose pump by spraying in through nozzles into salt-free water (3° C.). Ratio extract to salt-free water 1:3.
6) Allow to precipitate at 3° C. for 10 hours
7) Drawing off the clear supernate
8) Centrifuging the precipitated sediment for 5 minutes at 20000 g
9) Discarding the supernate
10) Precipitate: product according to the invention.

1-36. (canceled)

37. A protein preparation comprising protein particles, wherein each protein particle of said protein particles comprises a plurality of plant protein molecules with each of said protein molecules having at least one hydrophilic moiety and at least one hydrophobic moiety, and the protein molecules being bound to each other in such a manner that the protein particle has a substantially hydrophobic surface.

38. The protein preparation according to claim 37, wherein the protein molecules are bound to each other in such a manner that the protein particles have a rounded shape.

39. The protein preparation according to claim 38, wherein the rounded shape of the protein molecules is that of a sphere, and the at least one hydrophilic moiety of the protein molecules point into an interior of the sphere and the at least one hydrophobic moiety projects toward an outer side of the sphere.

40. The protein preparation according to claim 37, wherein protein content of the protein particles is greater than 75% by weight based on a dry mass of the protein particles.

41. The protein preparation according to claim 37, wherein the protein particles have an average particle size of less than 50 μm.

42. The protein preparation according to claim 37, wherein the protein particles have an average particle size of less than 10 μm.

43. The protein preparation according to claim 37, wherein the protein particles contain proteins of one or more raw plant materials.

44. The protein preparation according to claim 43, wherein the raw plant materials are leguminosae or oilseeds.

45. The protein preparation according to claim 43, wherein the raw plant materials are sunflower, soya, field bean, pea or lupin seeds.

46. The protein preparation according to claim 37, further comprising oil.

47. The protein preparation according to claim 46, wherein oil content of the preparation, in relation to a dry substance content, is greater than 1% by weight.

48. The protein preparation according to claim 46, wherein oil content of the preparation, in relation to a dry substance content, is between 5 and 20% by weight.

49. The protein preparation according to claim 46, wherein the oil is at least partially attached as a thin layer onto the substantially hydrophobic surface of the protein particles.

50. The protein preparation according to claim 49, wherein the oil at least partially acts to separate the protein particles.

51. The protein preparation according to claim 46, wherein the oil includes unsaturated fatty acids in an amount greater than 30% by weight.

52. The protein preparation according to claim 46, wherein the protein molecules and the oil originate from a common plant.

53. The protein preparation according to claim 46, further comprising lipophilic substances.

54. The protein preparation according to claim 46, wherein the protein particles are mixed with greater than 20% by weight of said oil, and contain only a residual water content of less than 10%.

55. The protein preparation according to claim 37, wherein the protein preparation is produced from seeds of Lupinus angustifolius.

56. The protein preparation according to claim 37, in combination with a foodstuff wherein said preparation is present in an amount sufficient to enrich said foodstuff with protein, said protein being present in a concentration of 1% to 30% of said foodstuff.

57. The protein preparation according to claim 56, wherein said foodstuff contains either no water or water in an amount of less than 10%.

58. The protein preparation according to claim 37, in combination with a moist substance or a suspension having a water content of 50 to 80% by weight and which is incorporatable into a foodstuff.

59. The protein preparation according to claim 58, wherein said preparation is present in an amount sufficient to reduce viscosity of said foodstuff.

60. A method for producing a protein preparation from plant proteins, in which each of the plant proteins has at least one hydrophobic moiety and at least one hydrophilic moiety comprising dissolving or suspending said plant proteins in a solvent having a first ionic strength and a first temperature, to obtain a solution or a suspension; precipitating the plant proteins from said solution or said suspension by simultaneously lowering the first temperature and the first ionic strength of the solvent so as to form protein particles having a substantially hydrophobic surface.

61. The method according to claim 60, wherein the solvent is aqueous.

62. The method according to claim 60, wherein the first ionic strength and the first temperature of the solution or the suspension are lowered by combining therewith a second solvent having a second temperature and a second ionic strength.

63. The method according to claim 62, wherein said combining is by spraying the solution or the suspension into the second solvent through nozzles or by mixing with the second solvent in a mixing section.

64. The method according to claim 60, wherein the first temperature is between 25° C. and 40° C. and is lowered to below 10° C.

65. The method according to claim 62, wherein the second temperature is less than 4° C.

66. The method according to claim 60, wherein the first ionic strength corresponds to that of a sodium chloride solution with a salt content of 1 to 4% by weight.

67. The method according to claim 60, wherein the first ionic strength is lowered by a factor of 2 to 50.

68. The method of claim 60, wherein the first ionic strength is lowered by a factor of 3 to 10.

69. The method according to claim 63, wherein said mixing is at a mixture ratio of the solution or of the suspension to the second solvent of 1:3 to 1:12.

70. The method according to claim 60, further comprising obtaining the plant proteins from non-comminuted or comminuted raw plant materials comprising:
optionally purifying the raw plant materials, said purifying including suspending the raw materials in water in an environment of poor protein solubility, and separating purified raw materials from undesired substances dissolved in water by mechanical separation,
extracting of the plant proteins from the raw materials or from the purified raw materials by suspension of the raw materials or of the purified raw materials in an extraction agent such that the plant proteins are dissolved in the extraction agent,
obtaining the plant proteins in a form of a protein extract including the extraction agent and the plant proteins dissolved therein by mechanical separation of insoluble components,
optionally further treating the protein extract by diluting or concentrating the plant proteins in the extract, or drying the extract, or isolating the plant proteins from the extract.

71. A method for obtaining plant proteins from raw plant materials containing protein and phenol with phenol depletion, in which the raw plant materials are obtained from non-comminuted or comminuted raw plant materials containing protein and phenol, said method comprising:
extracting the plant proteins by dissolving or suspending the raw plant materials in an extraction agent, in which the extraction agent has a pH value of less than 8.5 and an ionic strength which corresponds to a common salt content in the extraction agent of greater than 0.5% by weight, whereby a co-extraction of phenolic compounds is reduced compared with a salt-free extraction agent and whereby oxidation of co-extracted phenols is suppressed,
obtaining an extract containing protein, which also contains co-extracted, non-oxidized phenolic compounds, by mechanical separation of insoluble components,
physical separation of co-extracted, non-oxidized phenolic compounds from the extract containing protein.

72. The method according to claim 71, wherein before the extracting, a cleaning of the raw plant materials containing protein and phenol is carried out, in which the raw plant materials containing protein and phenol are suspended in water in an environment of poor protein solubility and purified raw plant materials containing protein and phenol are obtained by mechanical separation of undesired substances dissolved in the water.

73. The method according to claim 71, wherein either the extraction agent has an increased salt concentration of 3 to 20% by weight common salt, or the extraction agent is a sodium chloride solution with a concentration of 0.5 mol/l or of 1.5 mol/l.

74. The method according to claim 71, wherein oxidation of dissolved phenolic compounds is suppressed by setting the pH value of the extraction agent to a value of less than 7.0.

75. The method according to claim 71, wherein the extract containing protein undergoes a further treatment including either diluting or concentrating the proteins in the extract, or drying of the extract, or isolating the proteins from the extract.
